(19) United States
(12) Patent Application Publication
(10) Pub. No.: US 2018/0085226 A1
(43) Pub. Date: Mar. 29, 2018

(54) CONVERTIBLE ANATOMIC TO REVERSE TOTAL SHOULDER ARTHROPLASTY DEVICE

(71) Applicant: Keith Baumgarten, Sioux Falls, SD (US)

(72) Inventor: Keith Baumgarten, Sioux Falls, SD (US)

(21) Appl. No.: 15/714,330

(22) Filed: Sep. 25, 2017

Related U.S. Application Data
(60) Provisional application No. 62/399,863, filed on Sep. 26, 2016.

(51) Int. Cl. A61F 2/40

(52) U.S. Cl. CPC A61F 2/4081; A61F 2/4059; A61F 2002/30654; A61F 2002/4085; A61F 2/4003

(57) ABSTRACT

A convertible anatomic to reverse total shoulder arthroplasty device for utilizing a modular glenoid component in an anatomic shoulder arthroplasty facilitating later conversion to a reverse total shoulder arthroplasty when needed includes a baseplate for engaging and securing to a scapula. The baseplate includes a receiver extending into the baseplate. A glenoid component has a cavity section and a shaft section. The shaft section of the glenoid component is insertable into the receiver for replacing a glenoid fossa of the scapula. A humeral ball is couplable to a stem for being secured to a humerus wherein the humeral ball abuts and is engaged to the glenoid section of the glenoid component.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to joint arthroplasty devices and more particularly pertains to a new joint arthroplasty device for utilizing a modular glenoid component in an anatomic shoulder arthroplasty facilitating later conversion to a reverse total shoulder arthroplasty when needed.

### BRIEF SUMMARY OF THE INVENTION

An embodiment of the disclosure meets the needs presented above by generally comprising a baseplate for engaging and securing to a scapula. The baseplate includes a receiver extending into the baseplate. A glenoid component has a cavity section and a shaft section. The shaft section of the glenoid component is insertable into the receiver for replacing a glenoid fossa of the scapula. A humeral ball is couplable to a stem for being secured to a humerus wherein the humeral ball abuts and is engaged to the glenoid section of the glenoid component.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front view of a convertible anatomic to reverse total shoulder arthroplasty device according to an embodiment of the disclosure.

FIG. 2 is an exploded front view of an embodiment of the disclosure.

FIG. 3 is a partial exploded view of an a reverse arthroplasty embodiment of the disclosure.

FIG. 4 is a partial exploded view of an embodiment of the disclosure.

FIG. 5 is a side view of a humeral bearing tray of an embodiment of the disclosure.

### DETAILED DESCRIPTION OF THE INVENTION

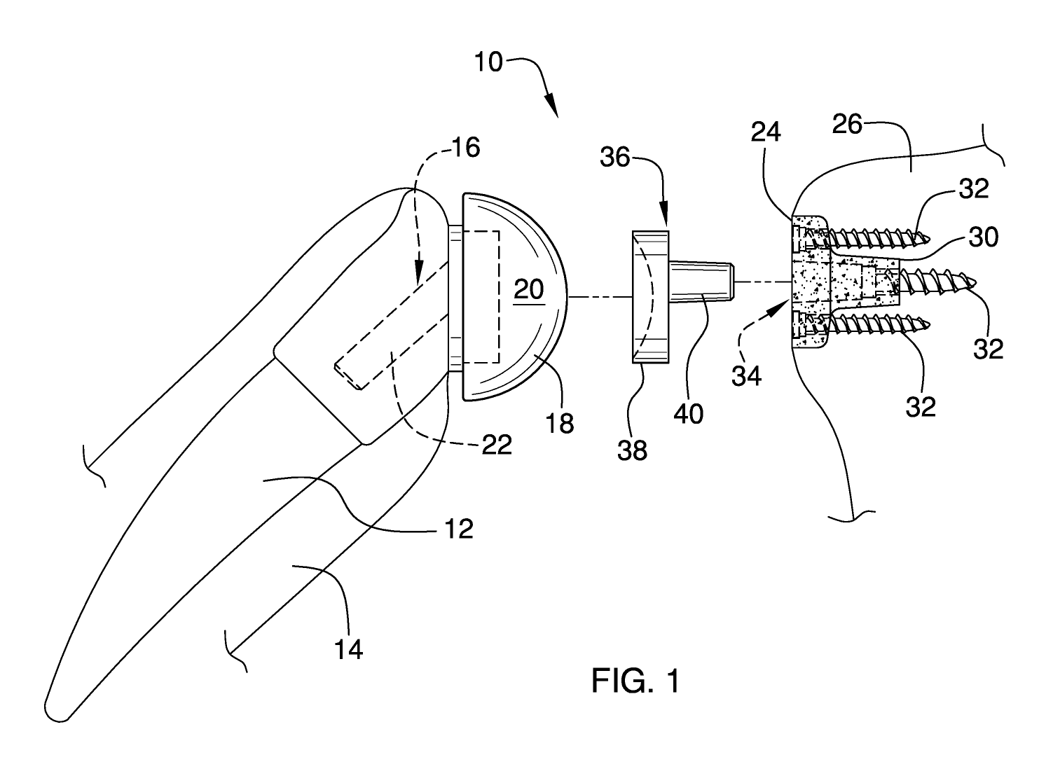

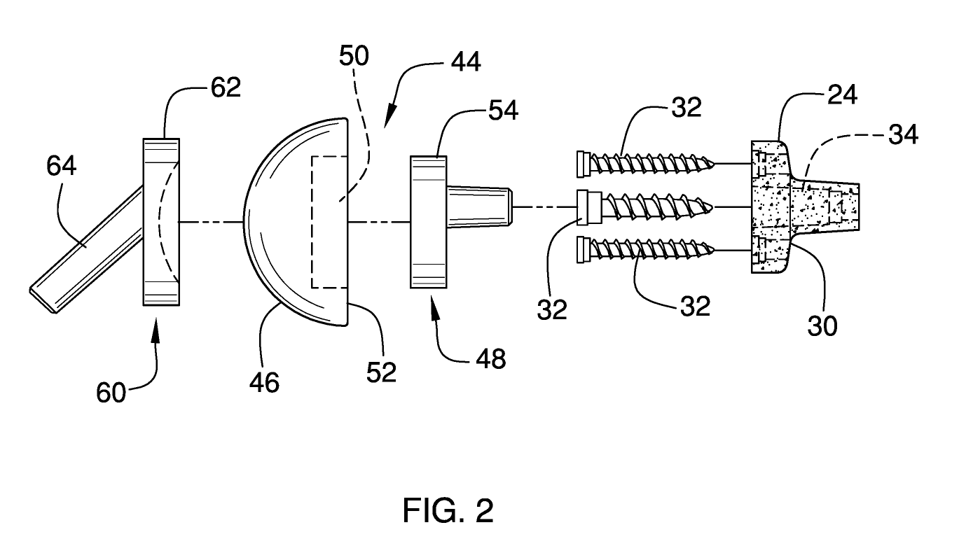

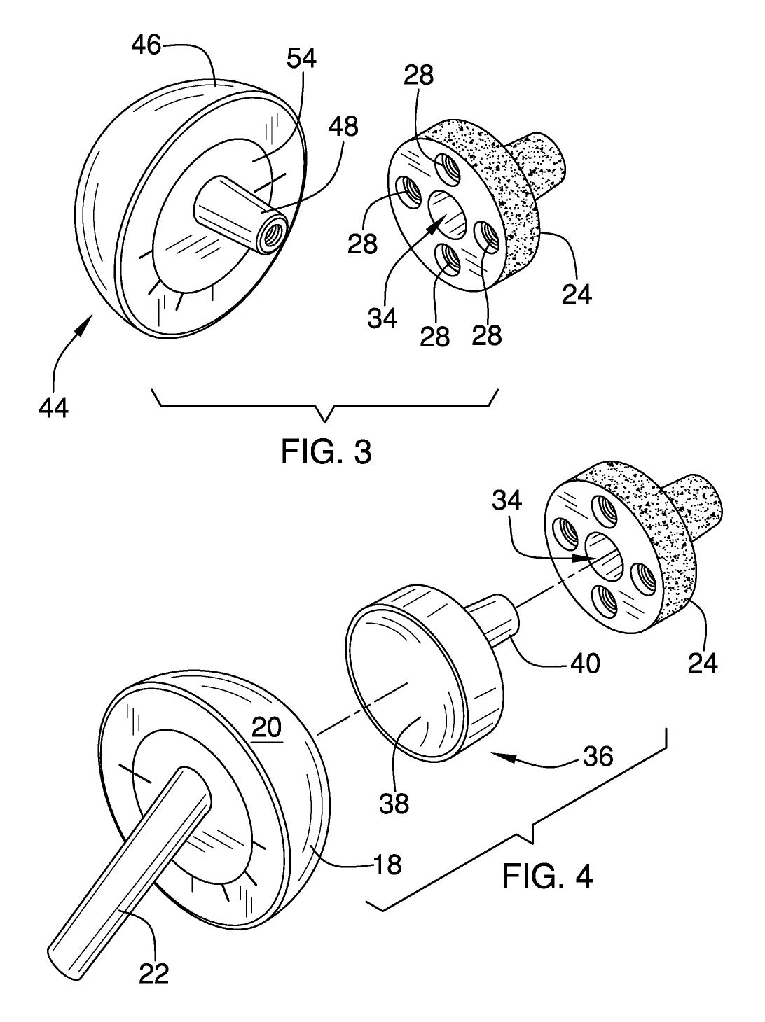

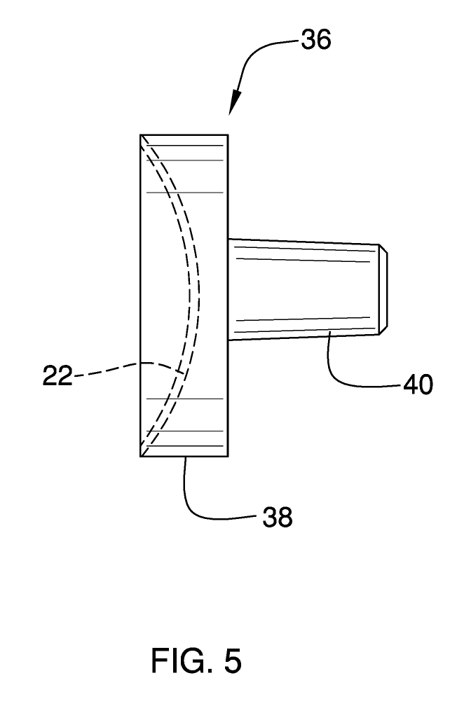

With reference now to the drawings, and in particular to FIGS. 1 through 5 thereof, a new joint arthroplasty device embodying the principles and concepts of an embodiment of the disclosure and generally designated by the reference numeral 10 will be described.

As best illustrated in FIGS. 1 through 5, the convertible anatomic to reverse total shoulder arthroplasty device 10 generally comprises a stem 12 is configured for engaging and securing to a humerus 14 in a conventional manner. The stem 12 includes a void 16 extending into the stem 12. A humeral ball 18 has a curved surface 20 and a post 22 extending outwardly away from the curved surface 20. The post 22 is complementary to the void 16 and insertable into the void 16 wherein the humeral ball 18 is configured for replacing a head of the humerus 14 as in a conventional anatomic shoulder arthroplasty. The post 22 of the humeral ball 18 is smooth and shaped complementary to the void 16 to be slidably insertable into the void 16. Thus, the humeral ball 18 is configured for being held in place relative to the stem 12 by anatomical compression in the shoulder after implantation. A humeral socket component or humeral bearing tray 60 has a socket part 62 and a post part 64. The post part 64 and the post 22 are similarly shaped and sized wherein the humeral ball 18 is replaceable by said humeral socket 60 component when converting an anatomic shoulder arthroplasty to a reverse arthroplasty.

A baseplate 24 is configured for engaging and securing to a scapula 26 in a conventional manner. The baseplate 24 has a plurality of connection holes 28 and a textured surface 30 to facilitate engagement of the baseplate 24 to the scapula 26. Screws 32 are used to secure the baseplate 24 to the scapula 26 in a conventional manner. The baseplate 24 further includes a receiver 34 extending into the baseplate 24. A glenoid component 36 has a cavity section 38 and a shaft section 40. The shaft section 40 of the glenoid component 36 is insertable into the receiver 34 wherein the glenoid component 36 is configured for replacing a glenoid fossa of the scapula 26. The shaft section 40 of the glenoid component 36 has a smooth exterior surface and is slidably insertable into the receiver 34 wherein the glenoid component 36. The glenoid component 36 is held in place relative to the baseplate 24 by anatomical compression within a shoulder when implanted known as a press fit using a morse-taper connection.

A glenosphere component 44 has a glenosphere part 46 and a shaft part 48. The shaft part 48 is shaped and sized similarly to the shaft section 40 of the glenoid component 36. Thus, the glenosphere component 44 and glenoid component 36 are interchangably insertable into the receiver 34 to facilitate replacement of the glenoid component 36 by the glenosphere component 44 in a subsequent surgical procedure in which the baseplate 24 remains secured to the scapula 26. The glenosphere part 46 is separable from the shaft part 48. The glenosphere part 46 has a recess 50 extending into a face 52 of the glenosphere part 46. The shaft part 48 includes a head portion 54 insertable into the recess 50 wherein the shaft part 48 and the glenosphere part 46 are joined to form the glenosphere component 44. The orientation of the head portion 54 within the recess 50 may alter or adjust the relative positioning of the shaft part 48 extending from the glenosphere part 46 in a conventional manner. The glenosphere component 44 is held in the joined state by anatomical compression within the shoulder after implantation.

The glenoid component 36 may be constructed of cobalt chromium (CoCr), a zirconium alloy, or a zirconia toughened alumina. The glenoid component 36 and the humeral ball 18 may be constructed of the same material. These materials are generally known for arthroplasty in the hip, but have not been applied to the shoulder.

Alternatively, the glenoid component 36 may comprise a polyethylene bearing surface layer 56 backed by metal which may be one of the above listed materials.

In use, the baseplate 24 and stem 12 are each secured to the scapula and humerus respectively using conventional methods. Anatomic arthroplasty is achieved by using the glenoid component 36 and humeral ball 18 combination being engaged to the baseplate 24 and stem 12. At a later time, such as after injury to a rotator cuff wherein a reverse shoulder arthroplasty may be deemed preferable, the anatomic arthroplasty may be converted to a reverse arthroplasty by replacement of the glenoid component 36 with the glenosphere component 44 combined with replacement of the humeral ball 18 by the humeral socket component 60. Simplified replacement of either the humeral ball 18 or the glenoid component 36 by a like piece may also be achieved in the event of failure, wear, or any other reason in which replacement may be desired. The replacements are achieved without having to remove or replace the baseplate 24 and stem 12 such that surgical time and resultant trauma to the patient is significantly reduced.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A total shoulder arthroplasty device comprising:
a baseplate, said base plate being configured for engaging and securing to a scapula, said baseplate including a receiver extending into said baseplate;
a glenoid component, said glenoid component having a cavity section and a shaft section, said shaft section of said glenoid component being insertable into said receiver wherein said glenoid component is configured for replacing a glenoid fossa of the scapula; and
a humeral ball couplable to a stem wherein said humeral ball is configured for being secured to a humerus wherein said humeral ball abuts and is engaged to said glenoid section of said glenoid component.

2. The device of claim 1, further comprising said shaft section of said glenoid component being slidably insertable into said receiver wherein said glenoid component is configured for being held in place relative to said baseplate by anatomical compression within a shoulder when implanted.

3. The device of claim 1, further comprising a glenosphere component, said glenosphere component having a glenosphere part and a shaft part, said shaft part being interchangably insertable into said receiver with said shaft section wherein said glenoid component is replaceable by said glenosphere component.

4. The device of claim 3, further comprising said glenosphere part being separable from said shaft part, said glenosphere part having a recess extending into a face of said glenosphere part, said shaft part including a head portion insertable into said recess wherein said shaft part and said glenosphere part are joined to form said glenosphere component.

5. The device of claim 1, further comprising said glenoid component being constructed of one material selected from the group of materials consisting of cobalt chromium (CoCr), a zirconium alloy, and a zirconia toughened alumina.

6. The device of claim 1, further comprising said humeral ball being constructed of one material selected from the group of materials consisting of cobalt chromium (CoCr), a zirconium alloy, and a zirconia toughened alumina.

7. The device of claim 1, further comprising both of said glenoid component and said humeral ball being constructed of one material selected from the group of materials consisting of cobalt chromium (CoCr), a zirconium alloy, and a zirconia toughened alumina.

8. The device of claim 1, further comprising said glenoid component and said humeral ball being constructed of the same material.

9. The device of claim 1, further comprising said glenoid component comprising polyethylene bearing surface layer backed by metal.

10. A total shoulder arthroplasty device comprising:
a stem, said stem being configured for engaging and securing to a humerus, said stem including a void extending into said stem;
a humeral ball, said humeral ball having a curved surface and a post extending outwardly away from said curved surface, said post being insertable into said void wherein said humeral ball is configured for replacing a head of the humerus; and
a glenoid component couplable to a baseplate wherein said glenoid component is configured for being secured to a scapula of the user wherein said glenoid component abuts and is engaged to said humeral ball.

11. The device of claim 10, further comprising said post of said humeral ball being slidably insertable into said void wherein said humeral ball is configured for being held in place relative to said stem by anatomical compression within a shoulder when implanted.

12. The device of claim 10, further comprising a humeral socket component, said humeral socket component having a socket part and a post part, said post part and said post being interchangably insertable into said void wherein said humeral ball is replaceable by said humeral socket component.

13. The device of claim 10, further comprising said glenoid component being constructed of one material selected from the group of materials consisting of cobalt chromium (CoCr), a zirconium alloy, and a zirconia toughened alumina.

14. The device of claim 10, further comprising said humeral ball being constructed of one material selected from the group of materials consisting of cobalt chromium (CoCr), a zirconium alloy, and a zirconia toughened alumina.

15. The device of claim 10, further comprising both of said glenoid component and said humeral ball being constructed of one material selected from the group of materials consisting of cobalt chromium (CoCr), a zirconium alloy, and a zirconia toughened alumina.

16. The device of claim 10, further comprising said glenoid component comprising polyethylene bearing surface layer backed by metal.

17. A total shoulder arthroplasty device comprising:
a stem, said stem being configured for engaging and securing to a humerus, said stem including a void extending into said stem;
a humeral ball, said humeral ball having a curved surface and a post extending outwardly away from said curved surface, said post being insertable into said void wherein said humeral ball is configured for replacing a head of the humerus, said post of said humeral ball being slidably insertable into said void wherein said humeral ball is configured for being held in place relative to said stem by anatomical compression;
a humeral socket component, said humeral socket component having a socket part and a post part, said post part and said post being interchangably insertable into said void wherein said humeral ball is replaceable by said humeral socket component;
a baseplate, said base plate being configured for engaging and securing to a scapula, said baseplate including a receiver extending into said baseplate;
a glenoid component, said glenoid component having a cavity section and a shaft section, said shaft section of said glenoid component being insertable into said receiver wherein said glenoid component is configured for replacing a glenoid fossa of the scapula, said shaft section of said glenoid component being slidably insertable into said receiver wherein said glenoid component is configured for being held in place relative to said baseplate by anatomical compression within a shoulder when implanted; and
a glenosphere component, said glenosphere component having a glenosphere part and a shaft part, said shaft part being interchangably insertable into said receiver with said shaft section wherein said glenoid component is replaceable by said glenosphere component, said glenosphere part being separable from said shaft part, said glenosphere part having a recess extending into a face of said glenosphere part, said shaft part including a head portion insertable into said recess wherein said shaft part and said glenosphere part are joined to form said glenosphere component.

18. The device of claim 18, further comprising:
said glenoid component being constructed of one material selected from the group of materials consisting of cobalt chromium (CoCr), a zirconium alloy, and a zirconia toughened alumina; and
said glenoid component and said humeral ball being constructed of the same material.

19. The device of claim 18, further comprising said glenoid component comprising polyethylene bearing surface layer backed by metal.
